(19) United States
(12) Patent Application Publication
(10) Pub. No.: US 2014/0294933 A1
(43) Pub. Date: Oct. 2, 2014

(54) COMPOSITION FOR DELIVERY OF ANGIOGENESIS INDUCING MOLECULE AND ARTIFICIAL TISSUE PREPARED BY THE COMPOSITION

(71) Applicant: SAMSUNG ELECTRONICS CO., LTD., Suwon-si (KR)

(72) Inventors: Jae Min CHA, Seoul (KR); Min Sang Kim, Anseong-si (KR); Hyun-Ryoung Kim, Guri-si (KR); Eun Sung Park, Seongnam-si (KR)

(73) Assignee: SAMSUNG ELECTRONICS CO., LTD., Suwon-si (KR)

(21) Appl. No.: 14/243,700

(22) Filed: Apr. 2, 2014

(30) Foreign Application Priority Data
Apr. 2, 2013 (KR) 10-2013-0036052

(51) Int. Cl. C12N 5/071; A61K 31/4035; A61K 47/48

(52) U.S. Cl. CPC C12N 5/0697; C12N 5/069; A61K 47/48823; A61K 31/4035
USPC 424/450; 435/373; 435/377; 514/417

(57) ABSTRACT

A composition for delivery of an angiogenesis inducing agent, including a temperature-sensitive liposome, a vascular cell-specific antibody, and an angiogenesis inducing agent, a tissue culture obtained using the composition, an artificial tissue including the tissue culture, and a method for preparing an artificial tissue are provided.

### CROSS-REFERENCE TO RELATED APPLICATIONS

This application claims the benefit of Korean Patent Application No. 10-2013-0036052 filed on Apr. 2, 2013 in the Korean Intellectual Property Office, the disclosure of which is hereby incorporated by reference.

### INCORPORATION-BY-REFERENCE OF MATERIAL ELECTRONICALLY SUBMITTED

Incorporated by reference in its entirety herein is a computer-readable nucleotide/amino acid sequence listing submitted herewith and identified as follows: 2,965 bytes ASCII (Text) file named “716352 ST25.TXT,” created Apr. 2, 2014.

### BACKGROUND OF THE INVENTION

1. Field of the Invention

Compositions and methods for delivery of an angiogenesis inducing agent to cells and/or tissues of interest are provided.

2. Description of the Related Art

When fabricated and cultured by tissue engineering, a three-dimensional artificial tissue with a thickness of 100 μm (micrometers) or more is less able to sufficiently receive adequate levels of oxygen and nutrients. Thus, cell necrosis is directly related to the depth of the tissue. Cell necrosis results in the loss of activity and functionality of the tissue.

To address this problem with conventional artificial tissues, a technique has been suggested in which an artificial tissue mimicking a bio-tissue having a vascular structure is fabricated by surface engineering a three-dimensional scaffold with an angiogenesis factor (Advanced Drug Delivery Reviews (2007) Volume 59, 249-262). However, since this technique is designed to induce angiogenesis in the scaffold prior to cell seeding, a competition for space and resources between the already-formed vessels and the subsequently seeded cells may undesirably result. In addition, the artificial tissue, although mimicking the vascular structure of natural bio-tissues, suffers from the disadvantage of being low in biocompatibility, which may prevent the introduction of the artificial tissue into a host.

Therefore, there is a need for compositions and methods for preparing an artificial tissue that not only allows for successful vascularization, but also is highly biocompatible.

### BRIEF SUMMARY OF THE INVENTION

Provided is a composition for delivery of an angiogenesis inducing agent, the composition including a temperature-sensitive liposome, a vascular cell-specific antibody, and an angiogenesis inducing agent.

Additionally, provided is a method for delivery of an angiogenesis inducing agent the method including contacting a cell of a tissue with a composition including a temperature-sensitive liposome, a vascular cell-specific antibody, and an angiogenesis inducing agent.

Another embodiment provides a tissue culture obtained by culturing a cell mixture of a vascular cell and a tissue cell together with the composition for delivery of an angiogenesis inducing agent.

A further embodiment provides an artificial tissue including the tissue culture.

Also provided is a method for preparing an artificial tissue including (a) culturing a cell mixture including a vascular cell and a tissue cell together with a temperature-sensitive liposome, a vascular cell-specific antibody, and an angiogenesis-inducing agent to obtain a cell culture; and (b) thermally treating the cell culture. Related compositions and methods are also provided.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a schematic drawing illustrating a process of conjugating an antibody to a temperature-sensitive liposome.

FIG. 2 is a schematic drawing illustrating the release mechanism of an angiogenesis-inducing agent through temperature-dependent destruction of the temperature-sensitive liposome in a composition for delivery of the angiogenesis-inducing agent as described herein.

FIG. 3 is a schematic drawing illustrating selective proliferation and selective differentiation of vascular cells in a 2-dimensional cell culture using a composition for delivery of the angiogenesis-inducing agent described herein.

FIG. 4 is a schematic drawing selective proliferation and selective differentiation of vascular cells in a 3-dimensional cell culture using the composition in accordance with one embodiment.

FIG. 5 is a schematic drawing of a vascularized cell culture or a 3-dimensional artificial tissue construct including the cell culture, prepared in accordance with methods described herein.

FIG. 6 consists of graphs displaying Fluorescence-Activated Cell Sorting (FACS) results of a control specimen which was not treated with immuno-liposomes.

FIG. 7 consists of graphs displaying FACS results using immuno-liposomes on CD31-positive HUVEC cells.

FIG. 8 consists of graphs displaying FACS results using immuno-liposomes on CD31-negative MCF-7 cells.

FIG. 9 consists of microscopic images of HUVEC and MCF-7 cells after treatment with immuno-liposomes.

### DESCRIPTION OF THE PREFERRED EMBODIMENTS

In tissue engineering, a 3-dimensional cell-based artificial tissue should be fabricated to ensure the high cellular activity fundamentally required by a tissue of interest, so that it can successfully function. In this context, a vascularization inducing system is provided for evenly delivering oxygen and nutrients deep to an artificial tissue construct.

According to one aspect, provided is a composition for delivery of an angiogenesis-inducing agent to a cell or tissue, the composition including a temperature-sensitive liposome, a vascular cell-specific antibody, and an angiogenesis-inducing agent. The vascular cell-specific antibody may be conjugated on the surface of the temperature-sensitive liposome, and the angiogenesis-inducing agent is encapsulated in the temperature-sensitive liposome.

Another embodiment provides a method for delivery of an angiogenesis inducing agent or for vascularization, the method including contacting a tissue cell with a mixture comprising a temperature-sensitive liposome, a vascular cell-specific antibody, and an angiogenesis inducing agent. The tissue cell may be a cell of a tissue in need of the delivery of the angiogenesis inducing agent or vascularization (i.e., formation of blood vessel). The method may further include a step of performing a thermal-treatment simultaneously with or after the contacting step. The tissue cell may be in a form of a cell mixture which further includes a vascular cell in addition to the cell derived from a tissue in need of the delivery of the angiogenesis inducing agent or vascularization. The contacting step may refer to a step of culturing the tissue cell or the cell mixture together with the mixture of a temperature-sensitive liposome, a vascular cell-specific antibody, and an angiogenesis inducing agent. The step of culturing may be performed as described with respect to a method of preparing an artificial tissue, below.

The term “tissue cell” may refer to a cell of a tissue of interest (e.g., other than a vascular cell) which is in need of angiogenesis (vascularization) (e.g., a tissue for the use in transplanting), or a cell (e.g., an embryonic stem cell, a pluripotent embryonic stem cell, etc.) which is capable of being differentiated to the tissue of interest. In particular, the tissue cell may be a cell derived (isolated) from a subject who needs transplantation of a tissue of interest. For example, the tissue of interest may be at least one tissue selected from the group consisting of internally vascularized (pre-vascularized) cardiac muscular tissue, bone tissue, cartilage tissue, muscular tissue, skin tissue, and any other suitable tissue. The term “vascular cell” may refer to any cell found in vasculatures, and for example, may be at least one cell type selected from the group consisting of vascular endothelial cells, smooth muscle cells, common myeloid progenitor cell, a cell (e.g., an embryonic stem cell, a pluripotent embryonic stem cell, etc.) capable of being differentiated to the at least one cell, and any other suitable cell type.

In order to more successfully perform delivery of an angiogenesis inducing agent or vascularization, the mixture of an angiogenesis inducing agent (e.g., PNF1) contained in the mixture of a temperature-sensitive liposome, a vascular cell-specific antibody, and an angiogenesis inducing agent may be used at a concentration of about 1 to about 100 μM (micromoles), for example, about 10 to about 50 μM, based on a total concentration of medium used in culturing the cell or cell mixture. The ratio between a temperature-sensitive liposome, a vascular cell-specific antibody, and an angiogenesis inducing agent in the mixture is as described below. The vascular cell contained in the cell mixture containing a vascular cell and a tissue cell may account for about 1 to about 30%, about 2 to about 20%, or about 5 to about 15% of the total cell number of the cells in the mixture. According to one embodiment, a pluripotent embryonic stem cell may be used as the cell mixture comprising a vascular cell and a tissue cell. In this embodiment, the pluripotent embryonic stem cell may be seeded at an initial density from about 1×10⁵ cells/ml to about 1×10⁹ cells/ml, or from about 1×10⁶ cells/ml to about 1×10⁸ cells/ml, for example, at an initial density of about 1×10⁷ cells/ml. The pluripotent embryonic stem cell may be cultured to form an embryoid body which is capable of being differentiated to a tissue of interest (e.g., cardiac muscular tissue, bone tissue, cartilage tissue, muscular tissue, skin tissue, etc.). Selective stimulation causing the spontaneous proliferation of vascular cells may be employed to adjust the number of the vascular cells to the aforementioned range (i.e., about 1 to about 30%, about 2 to about 20%, or about 5 to about 15%, based on the total cell number of the cell mixture).

The thermal treatment step is performed at a temperature that maintains the temperature of the cell culture at or higher than the phase transition temperature of the liposome, for example, at a temperature of about 38° C. to about 70° C., about 38° C. to about 60° C., about 38° C. to about 55° C., about 38° C. to about 45° C., or about 38° C. to about 42° C., about 39° C. to about 70° C., about 39° C. to about 60° C., about 39° C. to about 55° C., about 39° C. to about 45° C., or about 39° C. to about 42° C. The thermal treatment may be carried out for about 1 min to about 120 min, about 2 min to about 60 min, about 5 min to about 30 min, or about 7 min to about 15 min, simultaneously with the culturing step or about 10 min to about 300 min, about 60 min to about 200 min, about 90 min to about 150 min, or about 110 min to about 130 min after the culturing step. The thermal treatment may be performed by applying at least one stimulus selected from the group consisting of an ultrasonic wave (e.g., high intensity focused ultrasound, etc.), a magnetic field (e.g., an amplified magnetic field (AMF), etc.), a microwave, a high-frequency (radiofrequency), or any other suitable stimulus.

The temperature-sensitive liposome may include a lipid bilayer and a temperature-sensitive peptide (e.g., an elastin-like polypeptide (ELP), a leucine zipper, etc.) conjugated to a hydrophobic group-containing moiety. The temperature-sensitive liposome may further include a lipid bilayer stabilizer.

In the temperature-sensitive liposome, the hydrophobic group-containing moiety may be incorporated between the lipid molecules in which form the lipid bilayer, and the temperature-sensitive peptide conjugated thereto may be exposed outside or inside of the liposome.

As used herein, the term “lipid bilayer” refers to a membrane consisting of two layers of lipid molecules. The lipid bilayer may have similar thickness to that of a cell membrane present in nature. For instance, the lipid bilayer may have a thickness of about 10 nm or less, e.g., a thickness from about 1 nm to about 9 nm, from about 2 nm to about 8 nm, from about 2 nm to about 6 nm, from about 2 nm to about 4 nm, or from about 2.5 nm to about 3.5 nm.

A lipid bilayer may exhibit various phase transition behavior depending on temperature. That is, given a temperature, a lipid bilayer may be present in either a liquid or a gel (solid) phase. This is because lipid molecules consisting in the lipid bilayer have a characteristic temperature at which phase transition from gel phase to liquid phase occurs.

The term “phase transition temperature” (also known as melting temperature) refers to a temperature at which a material undergoes phase transition from solid phase to liquid phase or from liquid phase to solid phase.

The lipid bilayer may include a lipid molecule, for example, a phospholipid. As used herein, phospholipid refers to a molecule that has a hydrophobic head and two hydrophilic tails. When exposed to an aqueous phase, the phospholipids arrange themselves into a bilayer in which the hydrophobic tails face each other in inner side (core) of the bilayer and the hydrophilic phosphate heads face to the outside contacting to the aqueous phase on either side of the bilayer, thus forming a liposomal structure.

The lipid molecule may be at least one selected from the group consisting of any lipid molecules having a hydrophilic head and a hydrophobic tail. The lipid molecule may be at least one selected from the group consisting of phospholipids, sphingo lipids, glycolipids, and the like, all of which contain C12-C50 hydrocarbon chain (i.e., a hydrocarbon having 12 to 50 carbon atoms).

In one embodiment, the lipid molecule may be a phospholipid including a hydrocarbon of C12 to C22 or C14 to C20. The phospholipid may bear a saturated or unsaturated hydrocarbon with two acyl groups. The phospholipid may be at least one selected from the group consisting of phosphatidyl choline, phosphatidyl glycerol, phosphatidyl inositol, phosphatidyl ethanolamine, and the like. In addition, the phospholipid may have phase transition temperature ranging from about 10° C. to about 70° C., for example, from about 20° C. to about 65° C., from about 24° C. to about 55° C., from about 35° C. to about 45° C., from about 38° C. to about 45° C., from about 38° C. to about 42° C., from about 39° C. to about 45° C., or from about 39° C. to about 42° C. The phospholipid may be a mixture of two or more phospholipid molecules. When made of a mixture of two or more different phospholipids, the lipid bilayer may exhibit a wide spectrum of transition temperatures.

The phospholipid molecule may, for example, have two acyl groups, and may be at least one selected from the group consisting of C12 saturated chain phospholipid (e.g., Tc=critical temperature=about 10° C.), C14 saturated chain phospholipid (e.g., Tc=about 24° C.), C16 saturated chain phospholipid (e.g., Tc=about 41° C.), C18 saturated chain phospholipid (e.g., Tc =about 55° C.), C20 saturated chain phospholipid (e.g., Tc=about 65° C.), C22 saturated chain phospholipid (e.g., Tc=about 70° C.), or a combination thereof. An exemplary C16 saturated chain phospholipid may be dipalmitoylphosphatidyl choline (DPPC). DPPC is a saturated chain (C16) phospholipid with a phase transition temperature of about 41.5° C. The C18 saturated chain phospholipid may be exemplified by 1,2-distearoyl-sn-glycero-3-phosphocholine (DSPC). DSPC is a saturated chain (C18) phospholipid with a phase translation temperature of about 55.10° C. In one embodiment, the lipid molecule may be DPPC. In another embodiment, the phospholipid may be a mixture of DPPC and DSPC. The phospholipid may be a mixture of DPPC and DSPC in molar ratio (DPPC:DSPC) ranging from about 1:about 0 to about 0.5, or from about 1:about 0.1 to about 0.5.

The lipid bilayer may comprise at least one lipid selected from the group consisting of a sphingolipid such as a sphingomyelin, a glycolipid such as a ganglioside, or any other suitable lipids which have a phase transition temperature depending on its acyl chain length similarly to a phospholipid.

The lipid bilayer may include other membrane-forming materials besides a phospholipid. For example, lipid bilayer may include a material such as a bola lipid and/or a bacterial lipid which are capable of forming a solid-phase membrane. In addition, a block copolymer including a water-soluble polymer (e.g., polyethylene glycol, etc.) and a water-insoluble polymer (e.g., polypropylene oxide, polyethylethylene, etc.) may be employed.

At a phase transition temperature or higher, the lipid bilayer is disassembled with the lipid molecules entering a liquid phase. When composed of two or more different kinds of lipid molecules, the lipid bilayer is gradually disrupted starting from a lipid molecule having lower phase transition temperature. Hence, the disassembling temperature and disassembling degree of the lipid bilayer can be controlled by adjusting the composition of the lipid molecules of the lipid bilayer. The disassembly of the lipid bilayer at a phase transition temperature or higher leads to the release of the angiogenesis inducing agent encapsulated within the liposome. Accordingly, the release of the angiogenesis inducing agent encapsulated in the liposome can be controlled by temperature.

In one embodiment, the lipid bilayer may further include a lipid derivative, for example, a phospholipid derivative, which is derivatized (i.e., conjugated, chemically transformed, or substituted) with a hydrophilic polymer. The hydrophilic polymer may be at least one selected from the group consisting of polyethylene glycol (PEG), polylactic acid, polyglycolic acid, a copolymer of polylactic acid and polyglycolic acid, polyvinyl alcohol, polyvinyl pyrrolidone, oligosaccharides, and a combination thereof. The hydrophilic polymer may have an average molecular weight ranging from about 100 Da to about 100,000 Da. The lipid derivative with a hydrophilic polymer may be incorporated into the liposome in such a way that the lipid portions are located between lipid molecules of the lipid bilayer to be contained in the lipid bilayer as a component, with the hydrophilic polymer facing outside the liposome. The lipid derivative with a hydrophobic polymer may be composed of a phospholipid of C4-C30, for example, a phospholipid of C16-C24, to which PEG is conjugated. The derivative may be DPPC-PEG, or DSPE-PEG. In one embodiment, the PEG has a molecular weight of about 180 Da to about 50,000 Da.

The liposome may include a temperature-sensitive peptide conjugated to a hydrophobic group-containing moiety, wherein the hydrophobic group-containing moiety is incorporated into the lipid bilayer. As used herein the term “temperature-sensitive peptide” refers to an amino polymer which undergoes a conformation change depending on temperature. For example, it may be at least one selected from the group consisting of “elastin-like polypeptide (ELP)”, “leucine zipper motif”, “silk-like peptide”, and any other suitable peptides.

Any temperature-sensitive peptide may be utilized, so long as it undergoes changes in intramolecular or intermolecular hydrogen bonding with temperature changes, thereby leading to a conformational change. As the temperature increases, the temperature-sensitive peptide undergoes a conformational change from random coil to α-helix or β-turn, thereby contributing to the phase transition of the liposome. In one embodiment, the temperature-sensitive peptide may be at least one selected from the group consisting of an elastin-like polypeptide (ELP), a leucine zipper motif, a silk-like peptide, or a combination thereof. For example, the temperature-sensitive peptide may be an elastin-like polypeptide or a leucine zipper motif

Due to a hydrophobic effect, the hydrophobic group-containing moiety is intercalated between lipid molecules of the lipid bilayer, that is, located inside the lipid bilayer (i.e., regions in which hydrophobic tails of each layer of the lipid bilayer are gathered), playing a role in taking part in the constitution of the lipid bilayer and anchoring the conjugated temperature-sensitive peptide to the lipid bilayer. For example, the hydrophobic group-containing moiety may be a molecule with a hydrophobic trait. The hydrophobic group-containing moiety may be a lipid molecule which is the same as or different from a lipid molecule constituting the lipid bilayer.

The hydrophobic group-containing moiety may be at least one selected from the group consisting of a fully hydrophobic molecule and an amphipathic molecule including a hydrophobic portion and a hydrophilic portion. In the amphipathic molecule, its hydrophobic portion may be arranged inward of the lipid bilayer, while the hydrophilic portion directs outward of the lipid bilayer. The temperature-sensitive peptide may be conjugated to the hydrophilic portion, exposing it to the outside of the lipid bilayer (i.e., the internal space or the externally from the liposome). When the temperature-sensitive peptide is conjugated to the hydrophobic molecule, the hydrophobic molecule is arranged inwardly of the lipid bilayer while the temperature-sensitive peptide faces outward of the lipid bilayer, facing the outside of the liposome. Herein, the term “outward” refers to a direction from an inner portion (a portion of each layer where hydrophobic tails locate) of the lipid bilayer to an outer portion (a portion where hydrophilic heads locate) of the lipid bilayer (i.e., from hydrophobic tails to hydrophilic heads), thus, as used herein, “outward” may indicate a direction toward the internal space or the external environment of the liposome.

The hydrophobic group-containing moiety may be at least one selected from the group consisting of lipid molecules naturally existing in a biomembrane and lipid molecules which are not naturally found in a biomembrane but capable of forming a lipid bilayer.

The lipid naturally existing in a biomembrane may be at least one selected from the group consisting of phospholipid or its derivative, sterol or its derivative, sphingolipid or its derivative, and a combination thereof. The phospholipid or its derivatives may be at least one selected from the group consisting of phosphatidyl choline, phosphatidyl glycerol, phosphatidyl inositol, phosphatidyl ethanolamine, or a combination thereof. The sterol or its derivative may be at least one selected from the group consisting of cholesterol or its derivative and squalene or its derivative. The sphingolipid may be at least one selected from the group consisting of sphingomyelin or its derivative and ganglioside or its derivative. Examples of phospholipid, sterol, or sphingolipid used as the hydrophobic group-containing moiety may include an intermediate or a precursor produced during in vivo biosynthesis. The intermediate or precursor may be selected from the group consisting of phosphoglyceride, sphingosine, ceramide, cerebroside, and any other suitable intermediate or precursor.

In another embodiment, the hydrophobic group-containing moiety may be a saturated or unsaturated hydrocarbon, a saturated or unsaturated acyl molecule, or a saturated or unsaturated alkoxy molecule, all of which bear carbon atoms of C4-C30, C14-C24, or C16-C24.

The conjugation of the hydrophobic group-containing moiety and temperature-sensitive peptide may be effected by a linkage that is cleavable under a physiological or pathological condition. The linkage may be, for example, a cleavable linker depending on pH, a heat cleavable linker, a radiation cleavable linker, or a linker that is cleaved in aqueous solution.

In the hydrophobic group-containing moiety-conjugated to a temperature-sensitive peptide, the hydrophobic group-containing moiety may be conjugated to a side chain or a terminus (other than side chain) of the temperature-sensitive peptide. For example, the hydrophobic group-containing moiety may be bonded to the nitrogen atom (N) at the N-terminus or the carbonyl group (—C(O)—) at the C-terminus of the temperature-sensitive peptide. Alternatively, the conjugation between the hydrophobic group-containing moiety and the temperature-sensitive peptide may be accomplished by a linkage formed through a reaction with a functional group on the side chain of the peptide, wherein the functional group may be at least one selected from the group consisting of an amino group, a carbonyl group, a hydroxyl group, a thiol group, and a combination thereof. In addition, the hydrophobic group-containing moiety may be conjugated with the temperature-sensitive peptide through an amine or amide bond with a nitrogen atom (N) of the peptide. In another alternative embodiment, the hydrophobic group-containing moiety may be linked to a carbonyl group of C-terminus of the temperature-sensitive peptide via an amide or ester bond. In this regard, the hydrophobic group-containing moiety may include a single chain.

In the hydrophobic group-containing moiety, the hydrophobic portion may be an aliphatic hydrocarbon of C4-C30, for example, C14-C24 or C16-C24. The hydrophobic group-containing moiety may be at least one selected from the group consisting of myristoyl (C14), palmitoyl (C16), stearoyl (C18), arachidonyl (C20), behenoyl (C22), lignoceroyl (C24), and any other suitable C4-C30 hydrocarbon. Due to a hydrophobic effect, the hydrophobic group-containing moiety may be incorporated inside the lipid bilayer, so that the temperature-sensitive peptide conjugated to hydrophobic group-containing moiety can be anchored to the liposome.

In an embodiment, the temperature-sensitive peptide, such as elastin-like polypeptide, leucine zipper motif, and silk-like peptide, may be a polymer exhibiting an inverse phase transitioning behavior. As used herein, the term “inverse phase transitioning behavior” refers to an action by which a material is soluble in aqueous solutions at less than an inverse transition temperature (Tₜ), but becomes insoluble as the temperature is raised over Tₜ. With an increase in temperature, ELP transition may occur from an elongated chain state that is highly soluble to a tightly folded aggregate state which is greatly reduced in solubility. Such inverse phase transition may be induced because the conformation of elastin-like polypeptide is prone to change from β-turn to distorted β-structure as temperature increases. In some cases, elastin-like polypeptide may be defined on the basis of the temperature range in which the phase transition occurs. For example, the phase transition may be observed at a temperature from about 10° C. to about 70° C., from about 35° C. to about 45° C., from about 38° C. to about 45° C., from about 39° C. to about 45° C., from about 38° C. to about 42° C., or from about 39° C. to about 42° C.

When the temperature-sensitive peptide is linked to a component of the lipid bilayer, the inverse phase transitioning behavior may destroy the lipid bilayer due to shrinkage and self-assembly of the temperature-sensitive peptide as temperature rises from lower than Tₜ of the temperature-sensitive peptide to a higher temperature. When destroyed, the lipid bilayer may increase in transmittance. Thus, an active agent encapsulated within a liposome including the lipid bilayer may be released at higher yield (transmittance) from the liposome. However, the release of an active agent from the liposome is not limited to this particular mechanism.

The destruction of the lipid bilayer in a liposome by the inverse phase transitioning behavior of the temperature-sensitive peptide may vary depending on lipid molecules of the lipid bilayer, that is, the phase transition temperature of the lipid bilayer. A lipid bilayer may exist as a gel phase at lower than the phase transition temperature and as a liquid (crystalline) phase at higher than the phase transition temperature. For a gel phase, the destruction of the lipid bilayer may not occur or may be limited even though the temperature-sensitive peptide is conformationally changed to have changes to have a β-turn structure by the inverse phase transitioning behavior. However, when it exists as a liquid phase, the lipid bilayer may be apt to be destroyed as the temperature-sensitive peptide undergoes a conformation change to a β-turn structure due to the inverse phase transitioning behavior. That is, the inverse transition of the temperature-sensitive peptide is more likely to induce the destruction of the lipid bilayer which exists as a liquid phase than a gel phase. Therefore, a releasing temperature for an active agent encapsulated within in the liposome may be controlled by adjusting the phase transition temperature of a lipid bilayer of the liposome to coincide with the inverse phase transition temperature of the temperature-sensitive peptide.

As mentioned previously, the temperature, at which the angiogenesis inducing agent encapsulated within the temperature-sensitive liposome can be released, can be controlled by controlling the phase transition temperature of the lipid bilayer and/or the inverse phase transition temperature of the temperature-sensitive peptide in the liposome. For example, the phase transition temperature of the lipid bilayer or liposome including the temperature-sensitive peptide may be set within a range from about 10° C. to about 70° C., from about 10° C. to about 60° C., from about 10° C. to about 55° C., from about 10° C. to about 45° C., from about 20° C. to about 60° C., from about 20° C. to about 55° C., from about 30° C. to about 55° C., from about 30° C. to about 45° C., from about 35° C. to about 45° C., from about 38 to about 45° C., from about 39° C. to about 45° C., from about 38° C. to about 42° C., or from about 39° C. to about 42° C.

A liposome containing the temperature-sensitive peptide in accordance with an embodiment may be used to efficiently release an active agent encapsulated therein, compared to a liposome which does not contain the temperature-sensitive peptide, but a lipid bilayer alone. This is simply attributed to the fact that while the release of the active agent is induced by the dispersion of the lipid molecules (phase transition-induced destruction) in the liposome composed of a lipid bilayer alone, the release is further accelerated by the inverse phase transitioning behavior of the temperature-sensitive peptide, that is, by the destruction of the lipid bilayer due to shrinkage and assembly in the liposome containing the temperature-sensitive peptide. Herein, the active agent may be encapsulated in the interior space of the liposome, or in the interior of the lipid bilayer.

According to one embodiment, the elastin-like polypeptide may be defined by its amino acid sequence. For example, the elastin-like polypeptide may, partially or wholly, include one or more (for example, 1 to 200) repeating units wherein each repeating unit may be independently selected from the group consisting of VPGXG (SEQ ID NO: 1), PGXGV (SEQ ID NO: 2), GXGVP (SEQ ID NO: 3), XGVPG (SEQ ID NO: 4), GVPGX (SEQ ID NO: 5) or a combination thereof; where V stands for valine, P for proline, G for glycine, and X for any natural or non-natural amino acid except proline (e.g., each X is independently selected from the group consisting of alanine, isoleucine, leucine, methionine, phenylalanine, tryptophan, valine, asparagine, cysteine, glutamine, glycine, serine, threonine, tyrosine, aspartic acid, glutamic acid, arginine, histidine, and lysine). In one embodiment, X in each repeating unit may be independently valine or alanine

The repeating units may be separated by or terminate with one or more amino acids or other linker moieties while keeping the phase transition property of elastin-like polypeptide. For example, in embodiments where X comprises an amino acid having a side chain of an amine, e.g., lysine or arginine, X may be bonded at the terminal amine group on a side chain with a lipid or a linker which can be incorporated to the liposomal membrane.

The ratio of the repeating units to the amino acid having an amine group on its side chain or the linker moieties may be from about 0.1:about 99.9 to about 99.9:about 0.1, e.g., the ratio of the repeating units to the amino acid having an amine group on its side chain or the linker moieties may be about 10:about 90, about 20:about 80, about 30:about 70, about 50:about 50, about 60:about 40, about 75:about 25, about 99:about 1, or about 1:about 99. The repeating unit may be repeated two times or more, for example, about 2 to about 200 times.

In an embodiment, the elastin-like polypeptide may be a block polymer where VPGXG, PGXGV, GXGVP, XGVPG, GVPGX or a combination thereof is tandemly repeated once or more, or may include the block polymer. As long as the inverse phase transition behavior is maintained therein, the elastin-like polypeptide may be composed of VPGXG, PGXGV, GXGVP, XGVPG, GVPGX or combinations thereof and optionally another portion, for example the aforementioned linker and/or a blocking group. At the N- or C-terminus, the elastin-like polypeptide may be linked with a hydrophobic group-containing moiety. Also, a hydrophobic group-containing moiety may be conjugated to elastin-like polypeptide via a bond with a reactive or terminal group on the side chain of amino acid residues of the elastin-like polypeptide. The reactive group on the side chain may be an amino group, a hydroxyl group, a thiol group, or a carboxyl group.

The other terminus which remains unlinked with a hydrophobic group-containing moiety may be blocked (i.e., protected). For example, while a hydrophobic group-containing moiety is linked to the N-terminus of elastin-like polypeptide, the C-terminal carboxyl group of the elastin-like polypeptide may be blocked or remain unblocked. The blocking may be effected by linking or reacting with a material that may be biocompatible, non-immunogenic, helpful in a specific delivery, or able to avoid a biological degradation system. For example, the C-terminus may be blocked with an amino group via an amine linkage. The amino group may be an ammonia molecule, a primary amine, a secondary amine, or a tertiary amine. The primary, secondary, or tertiary amine may each have 1 to 10 carbon atoms, for example, 1 to 6 carbon atoms.

The repeating units may be each independently included in an elastin-like polypeptide and repeated one or more times. The number of repetitions for the repeating units may be independently an integer of 1 to about 200, 1 to about 100, 1 to about 80, 1 to about 60, 1 to about 40, 1 to 10, 1 to about 12, 1 to about 8, 1 to about 6, 2 to about 200, 2 to about 100, 2 to about 80, 2 to about 60, 2 to about 40, 2 to about 10, 2 to about 12, 2 to about 8, 2 to about 6, about 4 to about 100, about 8 to about 80, about 10 to about 60, about 12 to about 40, about 20 to about 40, about 4 to about 10, about 4 to about 8, or about 4 to about 6.

In one embodiment, the ELP conjugated with the hydrophobic group-containing moiety may be palmitoyl-(VPGXG)n or stearoyl-(VPGXG)n, where n is an integer of 1 to 12, for example 2 to 12 or 2 to 6.

The leucine zipper domain consists of at least one heptad repeat (7 amino acids; represented by abcdefg), characterized by the predominance of the common amino acid leucine at the d position and hydrophobic amino acids at the “a” and “b” positions. The leucine zipper turns to an α-helix conformation at the phase transition temperature, with “a” and “d” on one strand of helix accounting for the coiled coil. At higher than the phase transition temperature, the coiled-coil structure dissociates to form a disordered peptide.

The leucine zipper may be bonded at its N- or C-terminus with the hydrophobic group-containing moiety. Alternatively, the hydrophobic group-containing moiety may be linked to a reactive group or a terminal group on the side chain of the amino acid residues of the peptide. The reactive group on the side chain may be an amino group, a hydroxyl group, a thiol group or a carbonyl group. In the leucine zipper, the other terminus which remains unlinked with a hydrophobic group-containing moiety may be blocked or not. For example, while a hydrophobic group-containing moiety is linked to the N-terminus of leucine zipper, the C-terminal carboxyl group of the leucine zipper may be blocked or remain unblocked. The blocking may be effected by linking or reacting with a material that may be biocompatible, non-immunogenic, helpful in a specific delivery, or resistant against a biological degradation system. For example, the C-terminus may be blocked with an amino group via an amine linkage to the C-terminal carboxylic group of the leucine zipper. The amino group may be an ammonia molecule, a primary amine, a secondary amine, or a tertiary amine. The primary, secondary, or tertiary amine may each have 1 to 10 carbon atoms, for example, 1 to 6 carbon atoms.

The leucine zipper may be represented by [XSZLESK]n, in which [XSZLESK](SEQ ID NO: 6) is a repeating unit and n means the repetitions number of the repeating unit [XSZLESK], wherein the repeating units independently have valine (V) or lysine (K) for X, and serine (S) or lysine (K) for Z, and n corresponds to a number and may be an integer of 1 or higher. When repeated two or more times, the repeating units [XSZLESK] may be the same or different in amino acid sequence. The repeating units may be independently included in an ELP with one or more integer number of repetition. For example, the repeating number may be an integer ranging from 1 to about 200, from 1 to about 100, from 1 to about 80, from 1 to about 60, from 1 to about 40, from 1 to about 10, from 1 to about 12, from 1 to about 8, from 1 to about 6, from 2 to about 200, from 2 to about 100, from 2 to about 80, from 2 to about 60, from 2 to about 40, from 2 to about 12, from 2 to about 10, from 2 to about 8, from 2 to about 6, from about 4 to about 100, from about 8 to about 80, from about 10 to about 60, from about 12 to about 40, from about 20 to about 40, from about 4 to about 10, from about 4 to about 8, or from about 4 to about 6. In one embodiment, the leucine zipper may be [VSSLESKVSKLESKKSKLESKVSKLESKVSSLESK] (SEQ ID NO: 7)-NH₂.

In the liposome, a molar ratio of primary lipid molecules of the lipid bilayer (i.e., phospholipids) to hydrophobic group-containing moiety-conjugated temperature-sensitive peptide (i.e., ELP or leucine zipper) may be appropriately selected according to the properties of both the lipid bilayer and the hydrophobic group-containing moiety-conjugated temperature-sensitive peptide. For example, a molar ratio of primary lipid molecules: hydrophobic group-containing moiety-conjugated temperature-sensitive peptide may be about 50 to about 99.9:about 0.1 to about 50. For example, a molar ratio of primary lipid molecules (DPPC or mixtures of DPPC and DSPC) to hydrophobic group-containing moiety-conjugated ELP (palmitoyl(VPGXG)n or stearoyl(VPGXG)n, wherein n is an integer of 2 to 12) or leucine zipper (palmitoyl [XSZLESK]n or stearoyl [XSZLESK]n, wherein X is valine (V) or lysine (K), Y is serine (S) or lysine (K),and n means a repeating number and is an integer of 2 to 12) may be about 50 to about 99.0:about 0.1 to about 50.

The temperature-sensitive liposomes may further include a lipid bilayer stabilizing agent (lipid bilayer stabilizer). For a liposome including a temperature-sensitive peptide such as ELP or leucine zipper, a stabilizer for increasing the stability of the lipid bilayer, if introduced to the lipid bilayer, also plays a role in effectively releasing the active agent. The lipid bilayer stabilizer may be a lipid which has a phase transition temperature equivalent to or higher than that of the lipid bilayer. The lipid bilayer stabilizer may be at least one selected from the group consisting of steroids, glycolipids, sphingolipids, and derivatives thereof.

For instance, the lipid bilayer stabilizer may be a compound, e.g., a steroid compound, capable of being incorporated in (inside) the lipid bilayer. As used herein, the term “steroid” refers to an organic compound with a chemical structure that contains the core of gonane or a skeleton derived therefrom which takes the form of four fused rings: three cyclohexane rings (designated as rings A, B and C from the left to the right) and one cyclopentane ring (the D ring). Herein, “skeleton derived therefrom” refers to the introduction of an unsaturated bond to the gonane skeleton. The steroids vary by the functional groups attached to this four-ring core and by the oxidation state of the rings. For example, the steroids may include a hydrophilic functional group on the ring. For example, the steroids may have a hydroxyl group on the ring.

In one embodiment, the steroid may be a sterol. The sterol may be a special form of steroids, with a hydroxyl group at position-3 and a skeleton derived from cholestane. In the context of sterols, the term “skeleton derived from” refers to the introduction of an unsaturated bond to the cholestane skeleton. The steroids may be any of those found in plants, animals, and fungi. For example, they are may be synthesized from lanosterol as in animals and fungi, or from cycloartenol as in plants. The sterols may be cholesterols or their derivatives. Here, “derivative” means a variant of cholesterol which retains ability to be incorporated to the lipid bilayer.

According to one embodiment, the stabilizer is at least one selected from the group consisting of cholesterols, sitosterols, ergosterols, stigmasterols, 4,22-stigmastadien-3-ones, stigmasterol acetates, lanosterols, cycloartenols, and any other suitable stabilizer

The stabilizing agents, for example cholesterols, may help strengthen the lipid bilayer and reduce the transmittance of the liposome, so that they can allow for the stable existence of the liposome at normal body temperature.

For a liposome including a temperature-sensitive peptide, the lipid bilayer stabilizer, if introduced to the lipid bilayer, serves to increase the stability of the lipid bilayer as well as playing a role in effectively releasing the active agent. The temperature-sensitive liposome is highly advantageous in term of the efficiency of drug release because its disruption temperature can be adjusted to a narrow range of from about 38° C. to about 45° C., from about 39° C. to about 45° C., from about 38° C. to about 42° C., or from about 39° C. to about 42° C.

In order for the liposome to desirably function in an aqueous environment, the ingredients should be present with appropriate content ratios.

In a temperature-sensitive liposome, for example, the molar ratio of lipid bilayer (e.g., phospholipid) to hydrophobic group-containing moiety-conjugated temperature-sensitive peptide (e.g., ELP or leucine zipper) to phospholipids derivatized (chemically transformed or substituted) with a hydrophilic polymer may be about 50 about 99.9:about 0.1 about 50:about 0 about 10.

For a temperature-sensitive liposome further including a lipid bilayer stabilizer (e.g., cholesterol), the molar ratio of phospholipid to hydrophobic group-containing moiety-conjugated temperature-sensitive peptide to phospholipid derivatized (chemically transformed or substituted) with hydrophilic polymer to lipid bilayer stabilizer may be about 50 to about 99.9:about 0.1 to about 50:about 0 to about 10:about 0.1 to about 50, for example, about 50 to about 99.9:about 0.1 to about 50:about 0 to about 10:about 1 to about 30, or about 50 to about 99.9:about 0.1 to about 50:about 0 to about 10:about 1 to about20, or about 50 to about 99.9:about 0.1 to about 50:about 0 to about 10:about 1 to about 15, and particularly, about 50 to about 60:about 0.1 to about 1:about 0 to about 5:about 8 to about 12.

The temperature-sensitive liposomes may be unilamellar vesicles (SUV) or multivesiclular vesicles with a diameter of from 50 nm to 500 nm, for example, from 50 nm to 400 nm, from 50 nm to 300 nm, from 50 nm to 200 nm, from 100 nm to 500 nm, from 100 nm to 400 nm, from 100 nm to 300 nm, or from 100 nm to 200 nm

According to one embodiment, the temperature-sensitive liposome may include a phospholipid, a hydrophobic group-containing moiety-conjugated temperature-sensitive peptide, a phospholipid derivative with a hydrophobic polymer, and cholesterol. Each ingredient is as described above.

In one embodiment, the phospholipid may be DPPC alone or in combination with DSPC. In the phospholipid, DPPC may be mixed at a molar ratio of about 1:about 0 about 0.5, for example, about 1:about 0.1 about 0.5 with DSPC. In the hydrophobic group-containing moiety-conjugated ELP, an acyl group accounts for the hydrophobic group-containing moiety while the ELP half includes (VPGXG)n or (GVPGX)m wherein X is an amino acid except proline, and n or m is an integer of 1 or greater. In one embodiment, X may be valine or alanine, n may be 1 to 12, and m may be 1 to 12. The hydrophobic group-containing moiety-conjugated ELP may be stearoyl-(GVPGX)₂₋₆. The terminal carboxyl group of stearoyl-(GVPGX)₂₋₆. may be blocked or not. The C-terminus may be blocked with an amino group (e.g., ammonia) via an amine linkage to the C-terminal carboxylic group. The phospholipid derivatives with hydrophilic polymers may be DPPC-PEG or DSPE-PEG. The PEG may have a molecular weight of about 180 Da to about 50,000 Da.

In an alternative embodiment, the temperature-sensitive liposome may further include a cationic lipid. When the angiogenesis inducing agent is negatively charged, the cationic lipid may form a complex with the angiogenesis inducing agent by electrostatic interaction (attraction) so as to aid the encapsulation of the angiogenesis-inducing agent within the liposome. In addition, during the destruction of the lipid bilayer at the phase transition temperature or higher, the cationic lipid retains electrostatic attraction for the negatively charged angiogenesis-inducing agent, thereby preventing the burst release of the angiogenesis-inducing agent. That is, the cationic lipid is not only helpful for loading a negatively charged angiogenesis-inducing agent to the liposome, but also plays an important role in preventing the initial burst release of the agent during the destruction of the lipid bilayer at a target site (vascular cells) and thus in controlling the consistent release of the agent.

So long as it forms a complex with a negatively charged agent by electrostatic interaction, any cationic lipid may be employed. For Example, the cationic lipid may be at least one selected from the group consisting of 1,2-dipalmitoyl-3-trimethylammonium-propane (DPTAP), 1,2-dioleoyl-3-trimethylammonium-propane (DOTAP), N,N-dioleoyl-N,N-dimethylammonium chloride (DODAC), N,N-distearyl-N,N-dimethylammoniumbromide (DDAB), N-(1-(2,3-dioleoyloxy)propyl-N,N,N-trimethylammoniumchloride, N,N-dimethyl-(2,3-dioleoyloxy)propylamine (DODMA), N,N,N-trimethyl-(2,3-dioleoyloxy)propylamine (DOTMA), 1,2-diacyl-3-trimethylammonium-propane (TAP), 1,2-diacyl-3-dimethylammonium-propane (DAP), 3-beta-[N-(N′,N,N′-trimethylaminoethane)carbamoyl]cholesterol (TC-cholesterol), 3-beta[N-(N′,N-dimethylaminoethane)carbamoyl]cholesterol (DC-cholesterol), 3-beta[N-(N′-monomethylaminoethane)carbamoyl]cholesterol (MC-cholesterol), 3-beta[N-(aminoethane)carbamoyl]cholesterol (AC-cholesterol), cholesteryloxypropane-1-amine (COPA), N-(N′-aminoethane)carbamoylpropanonic tocopherol (AC-tocopherol), N-(N′-methylaminoethane)carbamoylpropanonic tocopherol (MC-tocopherol), and the like. In addition, the cationic lipid may be in the form of a lipid conjugate in which a peptide including about 1 to about 10, about 1 to about 9, about 3 to about9, or about 3 to about 5 cationic amino acids selected from the group consisting of arginine, histidine, lysine and a combination thereof is conjugated with a fatty acid of C12˜C22, C14˜C20, or C16˜C18.

In one embodiment, the cationic lipid may be a lipid conjugate in which DPTAP, DOTAP, or a peptide including about 1 to about 9 cationic amino acids (e.g., arginine) is conjugated with a fatty acid of C16˜C18 (e.g., stearic acid). When the temperature-sensitive liposome includes a cationic lipid, the molar ratio of phospholipid to cationic lipid in the lipid bilayer may be about 85 to about 99:about 1 to about 15 or about 90 to about 99:about 1 to about 10.

The vascular cell-specific antibody may be any antibody that recognizes and/or binds to a protein (e.g., receptor) specifically expressed on the membrane of vascular cells (e.g., vascular endothelial cells, etc.). The vascular cells for which the antibody is specific may be all cells that are found in a vasculature, for example, vascular endothelial cells. In one embodiment, the vascular cells are vascular endothelial cells while the protein specifically expressed in the cell membrane is selected from among CD31, CD144 (VE-Cadherin), CD102, CD106, Tie-2, and a combination thereof. The protein may be derived from mammals including primates, such as humans, monkeys, etc., and rodents such as rats, mice, rabbits, or any other suitable mammal.

The vascular cell-specific antibody may be selected from the group consisting of an anti-CD31 antibody, an anti-CD144 (VE-Cadherin) antibody, an anti-CD102 antibody, an anti-CD106 antibody, an anti-Tie-2 antibody, and a combination thereof.

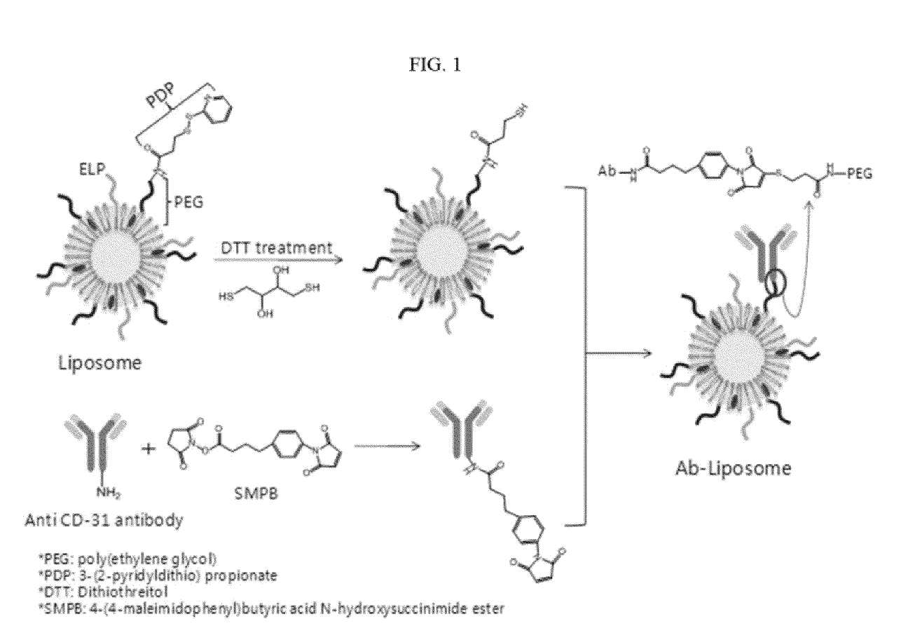

The vascular cell-specific antibody may be conjugated to the surface of the temperature-sensitive liposome. The conjugation of the vascular cell-specific antibody to the temperature-sensitive liposome may be effected through a chemical bond (e.g., amide bond) between functional groups. For example, the antibody may be conjugated to the liposome via a hydrophilic polymer stemming out of the liposomal surface, such as polyethylene glycol (PEG). As such, the surface modification of the liposome by conjugating an antibody to the surface via polyethylene glycol may lead to an increase in the targeting efficiency of the liposome. More particularly, functional groups, such as amine (—NH₂), thiol, maleimide, etc., are independently introduced to both the terminus of the hydrophilic polymer (e.g., PEG) stemming out of the surface of the liposome and the C-terminus of the heavy chain (Fc region) of the antibody, followed by forming an amide bond or thioether bond between the function groups through which the antibody can be conjugated to the surface of the liposome. A process of conjugating an antibody to a liposome in accordance with one embodiment is schematically shown in FIG. 1.

The angiogenesis-inducing agent may be at least one selected from the group consisting of angiogenesis-inducible small molecules (Mw; about 5,000 Da or less, e.g., about 100 to about 5,000 Da), such as a phthalimide neovascular factor (PNF) (e.g., PNF1), and vascular endothelial growth factors (VEGF; e.g., polypeptides translated from nucleotide sequences (mRNA) deposited under GenBank Accession Numbers NM_{—}001025366.2, NM_{—}001025367.2, NM_{—}001025368.2, NM_{—}001025369.2, NM_{—}001025370.2, NM_{—}001033756.2, NM_{—}001171622.1, NM_{—}001171623.1, NM_{—}001171624.1, NM_{—}001171625.1, NM_{—}001171626.1, NM_{—}001171627.1, NM_{—}001171628.1, NM_{—}001171629.1, NM_{—}001171630.1, NM_{—}001204384.1, NM_{—}001204385.1, NM_{—}003376.5, etc.), or a combination thereof.

PNF 1 is represented by the following Chemical Formula:

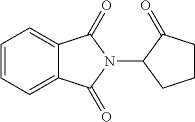 [SMILES: O=C1CCCC1N1C(=O)c2ccccc2C1=O]

Ratios between the temperature-sensitive liposome and the vascular cell-specific antibody can be adjusted depending on the composition and structure of the temperature-sensitive liposome and the kind of antibody. For example, the mass ratio of a temperature-sensitive liposome to an antibody may be adjusted from about 10 to about 1000:about 1, 50 to about 500:about 1, about 100 to about 400:about 1, or about 200 to about 300:about 1. In addition, the ratio between the temperature-sensitive liposome and the angiogenesis-inducing agent may be dependent on the composition and structure of the temperature-sensitive liposome and the kind of the angiogenesis-inducing agent. For example, the mass ratio of phospholipids in the temperature-sensitive liposome to the angiogenesis-inducing agent may be on the order of about 1 to about 50:about 1, about 1 to about 20:about 1, or about 5 to about 15:about 1.

The temperature-sensitive liposome may have a phase transition temperature ranging from about 10° C. to about 70° C., for example, from 10° C. to 60° C., from 10° C. to 55° C., from 10° C. to 45° C., from 20° C. to 60° C., from 20° C. to 55° C., from 20° C. to 45° C., from 30° C. to 60° C., from 30° C. to 55° C., from 30° C. to 45° C., from 35° C. to 45° C., from 38° C. to 45° C., from 38° C. to 42° C., from 39° C. to 45° C., or from 39° C. to 42° C. The phase transition temperature is affected by various factors including the carbon chain length, number of unsaturated bonds, and compositions of lipid molecules in the lipid bilayer. For example, DPPC in the mixture with DSPC may give the liposome a higher phase transition temperature than DPPC alone because DSPC is higher in melting temperature than DPPC. The temperature-sensitive liposome may be in a gel phase at room temperature.

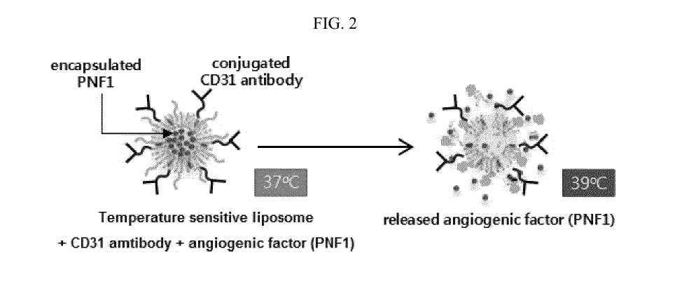

Because it is conjugated with vascular cell-specific antibodies at the surface thereof, the temperature-sensitive liposome is guided to specifically target vascular cells. At higher than the phase transition temperature of the lipid bilayer, the liposome is destroyed to release the angiogenesis-inducing agent encapsulated therein so that the angiogenesis-inducing agent can preferentially perform its activity on the vascular cells in contact with the liposome. This vascular cell-specific release of angiogenesis-inducing agents can bring about a more effective introduction of angiogenesis. FIG. 2 is a schematic view illustrating the release mechanism of an angiogenesis-inducing agent through the temperature-dependent destruction of the temperature-sensitive liposome in the composition for delivery of the angiogenesis-inducing agent in accordance with one embodiment.

Hence, when the composition including the temperature-sensitive liposome, the vascular cell-specific antibody, and the angiogenesis-inducing agent is incubated with a cell mixture of vascular cells and a tissue cell, the angiogenesis-inducing agent can temporarily act on the vascular cells with selectivity, generating a tissue culture in which vasculatures are successfully formed.

Another embodiment provides a tissue culture obtained by incubating a cell mixture of vascular cells and a tissue cell in the presence of the composition for delivery of an angiogenesis-inducing agent.

The term “vascular cell” may refer to any cell found in vasculatures, and for example, may be at least one selected from the group consisting of vascular endothelial cell, smooth muscle cell, common myeloid progenitor cell, and the like, or a cell (e.g., an embryonic stem cell, a pluripotent embryonic stem cell, etc.) capable of being differentiated to the at least one cell. The term “tissue cell” may refer to a cell of a tissue of interest which is in need of angiogenesis (vascularization) (e.g., a tissue for the use in transplanting), or a cell (e.g., an embryonic stem cell, a pluripotent embryonic stem cell, etc.) which is capable of being differentiated to the tissue of interest. In particular, the tissue cell may be a cell derived (isolated) from a subject who needs transplantation of a tissue of interest. For example, the tissue of interest may be at least one selected from the group consisting of internally vascularized (pre-vascularized) cardiac muscular tissue, bone tissue, cartilage tissue, muscular tissue, skin tissue, and the like. The tissue culture obtained may include at least one selected from the group consisting of internally vascularized (pre-vascularized) cardiac muscular tissue, bone tissue, cartilage tissue, muscular tissue, skin tissue, and the like.

In order to obtain a tissue culture in which successful vasculatures are formed, the composition for delivery of an angiogenesis-inducing agent may be used at a concentration of the angiogenesis inducing agent, e.g., PNF1, of about 1 to about 100 μM (micromoles), for example, about 10 to about 50 μM. The ratio between a temperature-sensitive liposome, a vascular cell-specific antibody, and an angiogenesis inducing agent in the composition for delivery of an angiogenesis-inducing agent is as described below. The vascular cells may account for about 1 to about 30%, about 2 to about 20%, or about 5 to about 15% of the total cell number of the cell mixture containing both vascular cells and a tissue cell.

According to one embodiment, a pluripotent embryonic stem cell may be used as the cell mixture of a vascular cell and a tissue cell. In this case, the pluripotent embryonic stem cell may be seeded at an initial density from about 1×10⁵ cells/ml to about 1×10⁹ cells/ml, or from about 1×10⁶ cells/ml to about 1×10⁸ cells/ml, for example, at an initial density of about 1×10⁷ cells/ml. The pluripotent embryonic stem cell is cultured to form an embryoid body which is capable of being differentiated to a tissue of interest (e.g., cardiac muscular tissue, bone tissue, cartilage tissue, muscular tissue, skin tissue, etc.). A selective stimulation of the spontaneous proliferation of vascular cells may adjust the number of the vascular cells to the aforementioned range (i.e., about 1 to about 30%, about 2 to about 20%, or about 5 to about 15%, based on the total cell number of the cell mixture).

Cell culturing may be performed under medium and culture conditions typically used for vascular cells and tissue cultures. For example, vascular cells may be cultured in an endothelial cell growth medium (EGM-2 bullet kit (Lonza)) at about 37° C. for an appropriate period of time dependent on the growth kinetic of the cells cultured. These culturing conditions may be appropriately controlled depending on kinds of both cultured cells and desired tissues.

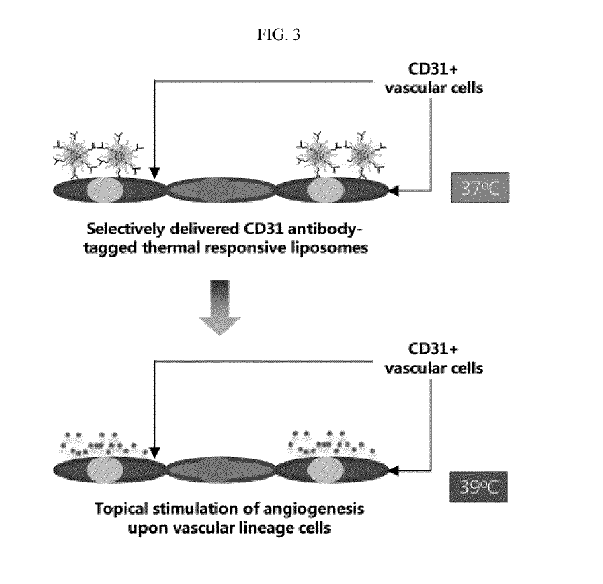

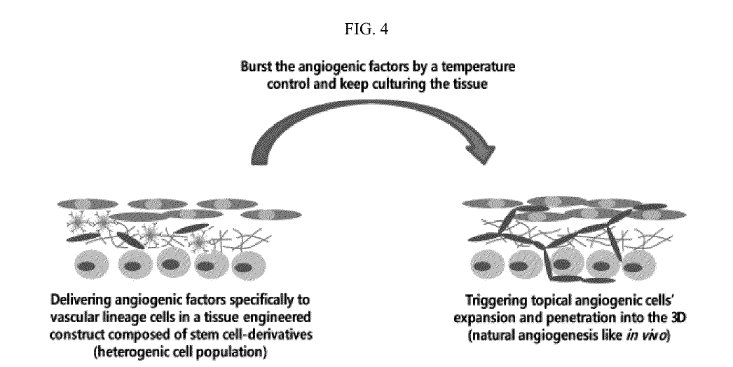

If the composition for delivery of an angiogenesis-inducing agent is heated to higher than the phase transition temperature of the temperature-sensitive liposome during the incubation, the temperature-sensitive liposome is destroyed, allowing the angiogenesis-inducing agent encapsulated within the temperature-sensitive liposome to perform its activity at a temporarily high concentration selectively on the vascular cells, which leads to forming vasculatures in the tissue culture at high efficiency. FIGS. 3 and 4 schematically illustrate the vascular cell-specific release and mechanism of an angiogenesis-inducing agent in a two- and a three-dimensional structure, respectively.

The tissue culture thus obtained may be a two- or three-dimensional construct. For example, when the cultured tissue cells are embryonic stem cells, the tissue culture may form a spheroid-like 3D tissue, known as an embryoid body. The tissue culture is supplied with necessary oxygen and nutrients and discharges waste products through the vasculatures successfully formed therein, thus preventing cell necrosis.

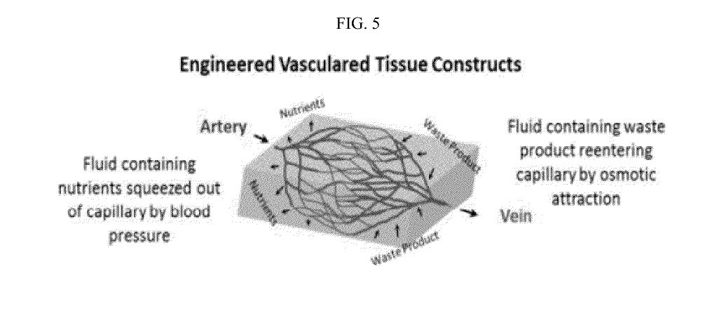

FIG. 5 is a schematic view of a cell culture, illustrating the life homeostasis therein.

This engineered tissue culture may be a complete tissue which is vascularized in a similar pattern to that of a biotissue, or a part of the complete tissue.

Accordingly, a further embodiment provides an artificial tissue including the tissue culture. The artificial tissue construct may be for bio-transplantation. The artificial tissue construct may be a two- or three-dimensional structure, and takes the form of a complete tissue, or a part of the complete tissue. Vascularized in the same pattern as in a bio-tissue, the artificial tissue construct can be transplanted to the body at a high success rate with reduced side effects.

Still another embodiment provides a method of preparing an artificial tissue, including (a) culturing a cell mixture comprising vascular cells and a tissue cell together with the composition for delivery of an angiogenesis-inducing agent (i.e., a mixture of a temperature-sensitive liposome, a vascular cell-specific antibody, and an angiogenesis-inducing agent) (culturing step), and (b) subjecting the cell culture to thermal treatment (thermal treatment step). In the artificial tissue prepared as above, vascularization is successfully achieved.

In the culturing step, the composition for delivery of an angiogenesis-inducing agent is used in such an amount that the concentration of the angiogenesis-inducing agent, e.g., PNF1 is on the order of 1 to 100 uM (micromoles), particularly on the order of 10 to 50 uM, per 1 mL of the medium while the vascular cells amounts to 1 to 30%, 2 to 20%, or 5 to 15% of the total cell number of the cell mixture, with the culturing temperature set forth at 35 to 38° C. Other conditions may be determined properly with reference to general experimental conditions for tissue formation. The culturing may be two- or three-dimensional cell culturing performed in a two- or three-dimensional culture vessel (e.g., a scaffold).

The thermal treatment step is to maintain the temperature of the cell culture at or higher than the phase transition temperature of the liposome, for example, at a temperature of about 38° C. to about 70° C., about 38° C. to about 60° C., about 38° C. to about 55° C., about 38° C. to about 45° C., or about 38° C. to about 42° C., about 39° C. to about 70° C., about 39° C. to about 60° C., about 39° C. to about 55° C., about 39° C. to about 45° C., or about 39° C. to about 42° C. The thermal treatment may be carried out for about 1 min to about 120 min, about 2 min to about 60 min, about 5 min to about 30 min, or about 7 min to about 15 min, simultaneously with the culturing step or about 10 min to about 300 min, about 60 min to about 200 min, about 90 min to about 150 min, or about 110 min to about 130 min after the culturing step. The thermal treatment may be performed by applying at least one selected from the group consisting of an ultrasonic wave (e.g., high intensity focused ultrasound, etc.), a magnetic field (e.g., an amplified magnetic field (AMF), etc.), a microwave, a high-frequency (radiofrequency), or any other suitable thermal treatment.

In one embodiment of the method, the cell mixture containing vascular cells and a tissue cell may be incubated in a two-dimensional culture vessel, and thermally heated to confirm the selective proliferation and differentiation of the vascular cells, followed by 3-dimensional, high-density tissue culturing to induce and promote 3-dimensional vascularization. In another embodiment, the cell mixture may include stem cells capable to be differentiated to various tissues, in addition to the vascular cells and a tissue cell.

In this method, the antibody contained in the composition for delivery of an angiogenesis-inducing agent specifically binds to the vascular cells, and the thermal treatment destroys the temperature-sensitive liposome, after or during which the angiogenesis inducing agent encapsulated within liposome is released to specifically act at a high concentration on the vascular cells, thus establishing an artificial tissue construct which is successfully vascularized.

The embodiments described above provide a solution to the fundamental problems with the conventional tissue engineering for fabricating 3-dimensional cell-based tissue construct, such as deficiency in nutrient and oxygen supply and resultant cell necrosis, making a needed contribution to the engineering of cell-based artificial tissue constructs which can be transplanted. Due to the composition for delivery of an angiogenesis-inducing agent and the method for fabricating a vascularized artificial tissue construct, the angiogenesis-inducing agent can be delivered specifically to vascular cells with the aid of the antibody specific for vasculatures (spatially controlled delivery).

Hereafter, the present invention will be described in detail by examples.

The following examples are intended merely to illustrate the invention and are not construed to restrict the invention in any way.

### EXAMPLES

### Example 1

### Construction of Temperature-Sensitive Liposome

Stearoyl-VPGVG VPGVG VPGVG-NH₂ (hereinafter referred to as “SA-V3-NH₂”), 1,2-distearoyl-sn-glycero-3-phosphocholine (DSPC), 1,2-dipalmitoyl-sn-glycero-3-phosphocholine (DPPC), 1,2-distearoyl-sn-glycero-3-phosphoethanolamine-N-[PDP (polyethylene glycol)-2000] (ammonium salt) (DSPE-PEG-PDP), and cholesterol were used at a molar ratio of 0.55:18.3:36.7:2:10 to construct a liposome in the form of unilamellar vesicles.

In further detail, SA-V3-NH₂ was dissolved at a concentration of 1 mg/mL in methanol, and DSPC, DPPC, DSPE-PEG-PDP, and cholesterol were each dissolved at a concentration of 10 mg/mL in chloroform. The methanol solution and the chloroform solution were mixed in a round-bottom flask, and the solvents were evaporated at room temperature from the mixture using a rotary evaporator to form lipid thin films on the inner wall of the flask.

The lipid thin films were hydrated by adding physiological saline (pH 7.3-7.6) at room temperature to the flask in such an amount as to make a phospholipid concentration of 10 mg/mL The hydrated solution was allowed to pass through a polycarbonate membrane with a pore size of 100 nm at room temperature to construct liposomes in the form of unilamallar vesicles.

### Example 2

### Conjugation of Antibody and Encapsulation of Angiogenesis Inducing Agent

An anti-CD31 antibody (ab9498, Abcam) was conjugated to the surface of the liposome constructed in Example 1 while PNF1 (phthalimide neovascular factor 1; Tissue engineering, 12, 1903, 2006) was encapsulated into the liposome.

The conjugation of the antibody was performed according to the procedure illustrated in FIG. 1. In further detail, the liposome constructed in Example 1 was incubated with 0.2 mL of 25 mM dithiothretol (DTT) for 30 min, and purified by passage through a desalting column (GE healthcare) to form an —SH group on the surface of the liposome. A solution of 0.075 mg of an anti-CD31 antibody (ab9498, Abcam) in 1 mL of PBS was reacted at room temperature for 30 min with 25 mM SMPB (4-(4-Maleimidophenyl)butyric acid N-hydroxysuccinimide ester) in DMF at a molar ratio of 1:20 antibody:SMPB to prepare an SMPB-conjugated anti-CD31 antibody which was then purified by passage through a desalting column. The liposome to the surface of which an —SH group was introduced was reacted at room temperature for 12 hrs with the SMPB-conjugated anti-CD31 antibody at a mass ratio of 250:1 liposome:antibody to give an antibody-conjugated liposome.

Meanwhile, PNF1 was dissolved at a concentration of 1 mg/mL in methanol during the construction of the liposome in Example 1 and then, evaporated together with the methanol solution and the chloroform solution to achieve the encapsulation of PNF1 within the liposome. At this time, the mass ratio of phospholipid to PNF1 was set forth as 10:1.

As a result, a temperature-sensitive liposome complex which had an anti-CD31 antibody conjugated to the surface thereof, and PNF1 encapsulated therein was prepared.

### Example 3

### Test for Targeting Vascular Cell

The anti-CD31 antibody-conjugated temperature-sensitive liposome was tested for targeting ability by measuring the antibody affinity for vascular cells.

In further detail, using the procedure described in Example 2, the temperature-sensitive liposome was conjugated with an anti-CD31 antibody (ab9498, Abcam) and a fluorescent (DPPE-cy5.5), instead of PNF1, was loaded in an amount of 1 wt % based on the total weight of the liposome to afford an immune-liposome having a fluorescent encapsulated therein.

The fluorescent-encapsulated, immune-liposomes were mixed at a concentration of 30 ug/ml with 2 mL of a cell culture medium and incubated with 3×10⁵ HUVEC cells (endothelial cell: CD31 positive cell; ATCC) or 3×10⁵ MCF-7 cells (epithelial cell: CD31 negative cell; ATCC). The affinity of the antibody was determined using FACS (Fluorescence Activated Cell Sorting) and a confocal microscope. The antibody-conjugated liposomes were associated with each of the cells, and the extra-liposomes which were not bound to the cells were washed off A single cell suspension was obtained in 0.05% (w/v) trypsin, and FACS was performed by measuring fluorescence with laser suitable to the cy5.5 dye.

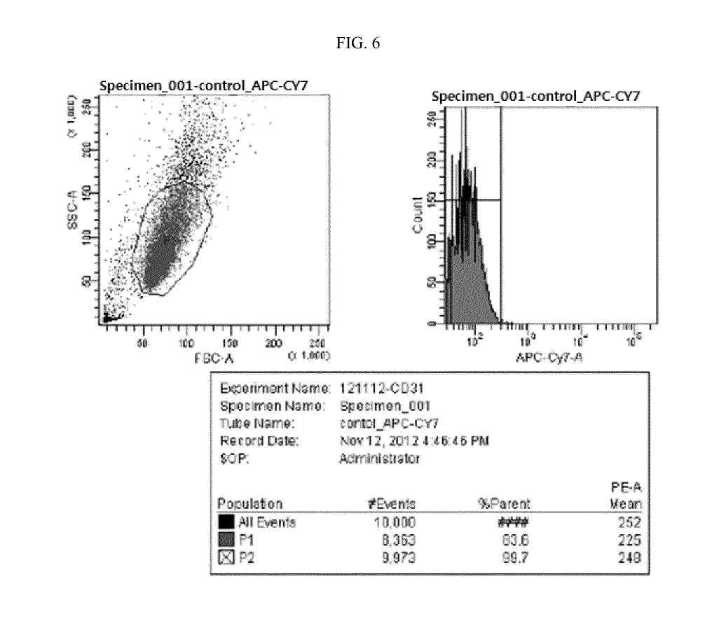

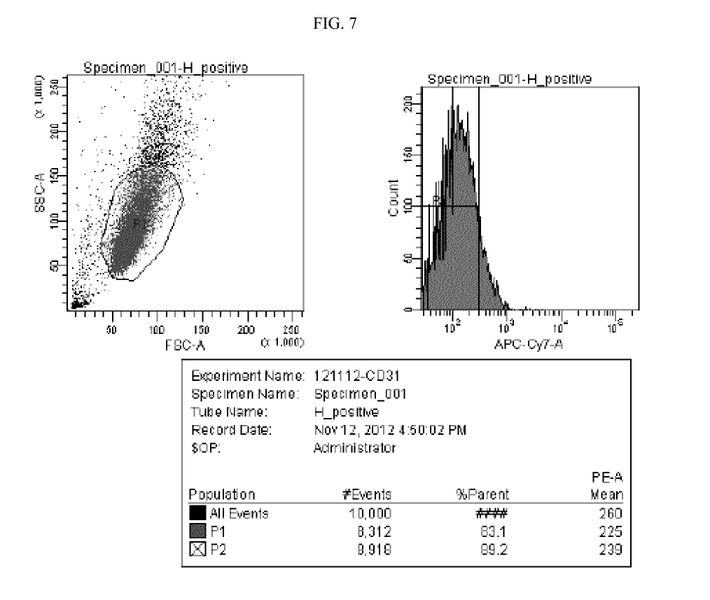

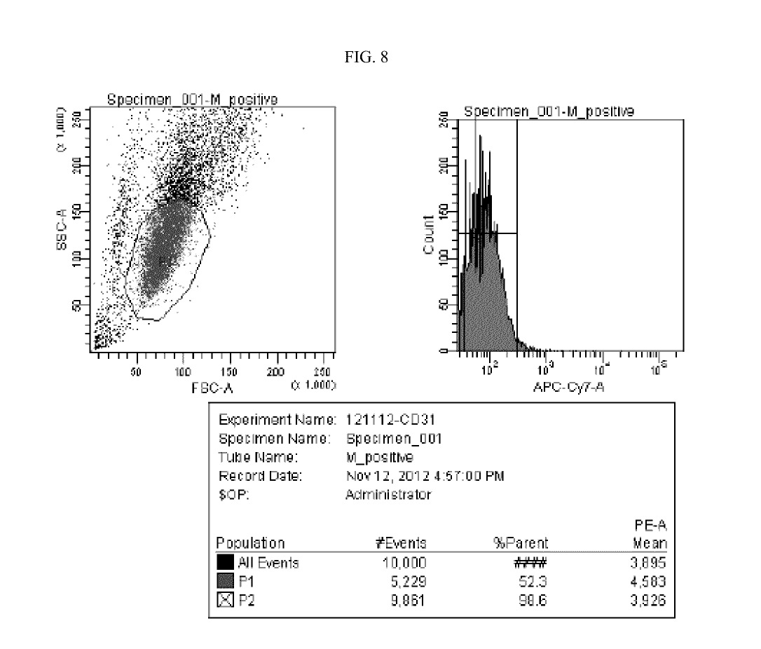

FACS results are given in FIGS. 6 to 8. The FACS results were obtained from a control which was not treated with the immune-liposomes in FIG. 6, from CD31-positive HUVEC in FIG. 7, and from CD31-negative MCF-7 cells in FIG. 8. As can be seen in FIGS. 6 to 8, a higher population of the immune-liposomes was detected in CD31-positive HUVEC than CD31-negative MCF-7.

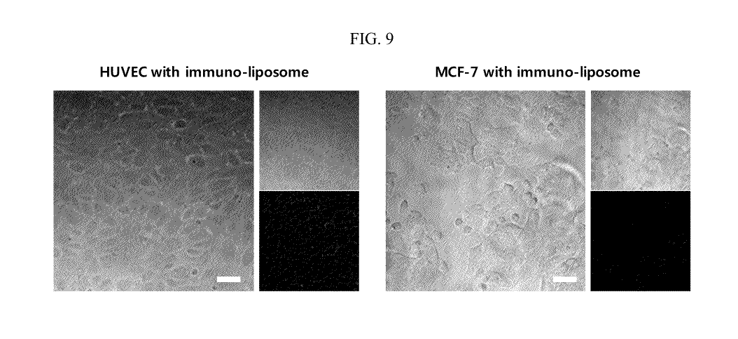

Confocal microscopic images of the cells are given in FIG. 9 (size bars: 40 μm). As can be seen in FIG. 9, almost no immune-liposomes were detected from CD31-negative MCF-7 whereas CD31 positive HUVEC was observed to have a large amount of the immuno-liposomes bound thereto, which was consistent with the FACS results. Particularly, the immuno-liposomes were found predominantly at the border of the membrane of HUVEC, indicating that the targeting affinity of the immuno-liposomes was attributed to the specific binding of the anti-CD31 antibody to the cell membrane.

### Example 4

### Fabrication of Artificial Tissue Construct

An artificial tissue construct was fabricated by incubating a cell mixture of vascular cells and other cells differentiated from stem cells with the temperature-sensitive liposome complex prepared in Example 2.

In further detail, E14Tg2a (murine embryonic stem cell line, ATCC) was cultured in high glucose Dulbecco's Modified Eagles Medium (DMEM; Invitrogen) (supplemented with 10% (v/v) ESC qualified-fetal bovine serum (FBS) (Invitrogen), 100 U/mL penicillin, 100 ug (microgram)/mL streptomycin (Invitrogen), 1 mM L-glutamine (Invitrogen), 0.1 uM (micromole) beta-mercaptoethanol (Sigma), and 1,000 U/mL leukemia inhibitory factor (LIF; Chemicon)) in a 0.1% (w/v) gelatin (Sigma)-coated tissue culture flask. The culturing was performed according to a procedure in which embryonic stem cells are expanded to a population sufficient for tissue formation while retaining pluripotency (stemness). Briefly, about 100,000 E14Tg2a cells were seeded to the cell culture flask with an area of 75 cm², and cultured for 2-3 days at 37° C. in a 5% CO₂ incubator. When reaching 70% confluency, the cells were subcultured at the same density in a new flask.

The cell mixture including vascular cells differentiated from stem cells was obtained by culturing an embryoid body. Embryonic stem cells (CRL-1821, ATCC) were single cell suspended in bacterial grade non-tissue culture treated petri-dishes to induce spontaneous tissue formation. In further detail, about 100,000 embryonic stem cells (CRL-1821, ATCC) were seeded to a cell culture flask with an area of 75cm² and incubated at 37° C. in a 5% CO2 incubator. At 70% confluency, trypsinization (0.05% Trypsin, Invitrogen) was conducted, followed by pipetting to establish single cell suspensions which were then applied to bacterial grade petri-dishes to allow for the spontaneous formation of embryoid bodies. These embryoid bodies were cultured in alpha Minimal Essential Medium (MEM; Invitrogen) (15% heat-inactivated FBS, 100 U/mL penicillin and 100 μg/mL streptomycin (Invitrogen)), with the exchange of the medium with a new one daily.

On day 5 after the embryoid bodies were suspended, the temperature-sensitive liposome complex which had the anti-CD31 antibody conjugated to the surface thereof and the vascularization-stimulating drug (PNF 1) encapsulated therein was added to the culture medium. The amount of the temperature-sensitive liposome complex was set forth such that PNF1 could be delivered at a concentration of 10-50 μM to each culture vessel.

For about 2 hrs after treatment with the temperature-sensitive liposome, the cells were incubated in a CO₂ incubator, and then washed out the liposomes which were not antibody-specifically bound to vascular cells before thermal treatment for about 10 min at 39° C. This thermal treatment destroyed the temperature-sensitive liposomes that were specifically bound to the vascular cells to release the PNF1 encapsulated within the temperature-sensitive liposome. As a result, since the temperature-sensitive liposome was associated with the vascular cells via the antibody conjugated to the surface thereof, the effect of PNF1 could be focused on the vascular cells.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A method of preparing an artificial tissue comprising
(a) culturing a cell mixture comprising a vascular cell and a tissue cell together with a temperature-sensitive liposome, a vascular cell-specific antibody, and an angiogenesis-inducing agent to obtain a cell culture; and
(b) subjecting the cell culture to a thermal treatment at a temperature equal to or greater than the phase transition temperature of the liposome.

2. The method of claim 1, wherein the temperature-sensitive liposome comprises:
a lipid bilayer and
a temperature-sensitive peptide conjugated to a hydrophobic group-containing moiety.

3. The method of claim 2, wherein the temperature-sensitive liposome further comprises a lipid bilayer stabilizer incorporated in the lipid bilayer of the liposome, and wherein the lipid bilayer stabilizer comprises at least one compound selected from the group consisting of steroids, glycolipids, and sphingolipids, or any combination thereof.

4. The method of claim 3, wherein the lipid bilayer stabilizer comprises at least one steroid, and the steroid is selected from the group consisting of cholesterols, sitosterols, ergosterols, stigmasterols, 4,22-stigmastadien-3-ones, stigmasterol acetates, lanosterols, and cycloartenols.

5. The method of claim 2, wherein the temperature-sensitive peptide comprises at least one of an elastin-like polypeptide or a leucine zipper.

6. The method of claim 5, wherein the temperature sensitive peptide comprises an elastin-like polypeptide, and the elastin-like polypeptide comprises 1 to 200 repeating units, wherein each repeating unit is independently selected from the group consisting the amino acid sequences of SEQ ID NO: 1, SEQ ID NO: 2, SEQ ID NO: 3, SEQ ID NO: 4, SEQ ID NO: 5 and a combination thereof.

7. The method of claim 5, wherein the temperature sensitive peptide comprises a leucine zipper, and the leucine zipper is represented by the sequence [XSZLESK]ₙ, wherein [XSZLESK] is a repeating unit, X in each repeating unit is independently valine (V) or lysine (K), Z in each repeating unit is independently serine (S) or lysine (K), and n is an integer ranging from 1 to 200.

8. The method of claim 2, wherein the hydrophobic group-containing moiety comprises a phospholipid, a sterol, a sphingolipid, a saturated or unsaturated hydrocarbon of C4-C30, a saturated or unsaturated acyl molecule of C4-C30, or a saturated or unsaturated alkoxy molecule of C4-C30.

9. The method of claim 2, wherein the lipid bilayer comprises a C12-C22 phospholipid.

10. The method of claim 2, wherein the lipid bilayer comprises at least one selected from the group consisting of phosphatidyl choline, phosphatidyl glycerol, phosphatidyl inositol, and phosphatidyl ethanolamine.

11. The method of claim 2, wherein the lipid bilayer further comprises a phospholipid which is chemically transformed with a hydrophilic polymer.

12. The method of claim 11, wherein the hydrophilic polymer is selected from the group consisting of polyethylene glycol, polylactic acid, polyglycolic acid, a copolymer of polylactic acid and polyglycolic acid, polyvinyl alcohol, polyvinyl pyrrolidone, an oligosaccharide, or a combination thereof.

13. The method of claim 1, wherein the temperature-sensitive liposome has a phase transition temperature of about 38° C. to about 45° C.

14. The method of claim 1, wherein the vascular cell-specific antibody comprises an antibody capable of binding to CD31, CD144 (VE-Cadherin), CD102, CD106, or Tie-2.

15. The method of claim 1, wherein the angiogenesis-inducing agent comprises at least one selected from the group consisting of an angiogenesis-inducible small molecule having a molecular weight of about 100 Da to about 5,000 Da, and a vascular endothelial growth factor (VEGF).

16. The method of claim 15, wherein the angiogenesis-inducing agent comprises a phthalimide neovascular factor.

17. The method of claim 1, wherein the vascular cell is selected from the group consisting of a vascular endothelial cell, a smooth muscle cell, a common myeloid progenitor cell, or a stem cell capable of differentiation.

18. The method of claim 1, wherein the thermal treatment is performed at a temperature of about 38° C. to about 45° C.

19. An artificial tissue prepared by the method of claim 1, wherein the artificial tissue comprises a culture which is obtained by culturing a cell mixture comprising a vascular cell and a tissue cell together with a temperature-sensitive liposome, a vascular cell-specific antibody, and an angiogenesis-inducing agent.

20. A method for delivery of an angiogenesis inducing agent, comprising contacting a tissue cell with a mixture comprising a temperature-sensitive liposome, a vascular cell-specific antibody, and an angiogenesis inducing agent, wherein the vascular cell-specific antibody is conjugated to the surface of the temperature-sensitive liposome and the angiogenesis-inducing agent is encapsulated in the temperature-sensitive liposome.

21. A composition comprising a temperature-sensitive liposome, a vascular cell-specific antibody, and an angiogenesis-inducing agent.


SEQUENCE LISTING (7 sequences)

SEQ ID NO: 1 - protein, 5 aa, Artificial Sequence
  Synthetic (unit of elastin-like polypetide, wherein Xaa is any amino acid except proline)
VPGXG

SEQ ID NO: 2 - protein, 5 aa, Artificial Sequence
  Synthetic (unit of elastin-like polypetide, wherein Xaa is any amino acid except proline)
PGXGV

SEQ ID NO: 3 - protein, 5 aa, Artificial Sequence
  Synthetic (unit of elastin-like polypetide, wherein Xaa is any amino acid except proline)
GXGVP

SEQ ID NO: 4 - protein, 5 aa, Artificial Sequence
  Synthetic (unit of elastin-like polypetide, wherein Xaa is any amino acid except proline)
XGVPG

SEQ ID NO: 5 - protein, 5 aa, Artificial Sequence
  Synthetic (unit of elastin-like polypetide, wherein Xaa is any amino acid except proline)
GVPGX

SEQ ID NO: 6 - protein, 7 aa, Artificial Sequence
  Synthetic (unit of Leucine zipper, wherein Xaa at 1st position is Val or Lys and Xaa at 3rd position is Ser or Lys)
XSXLESK

SEQ ID NO: 7 - protein, 35 aa, Artificial Sequence
  Synthetic (Leucin zipper)
VSSLESKVSKLESKKSKLESKVSKLESKVSSLESK